(19) United States
(12) Patent Application Publication
(10) Pub. No.: US 2026/0042088 A1
(43) Pub. Date: Feb. 12, 2026

(54) METHODS FOR PREPARING C2 TO C4 HYDROCARBONS AND SELF-BOUND HYBRID CATALYSTS

(71) Applicants: Dow Global Technologies LLC, Midland, MI (US); Dow Silicones Corporation, Midland, MI (US)

(72) Inventors: Florian Geyer, Wiesbaden (DE); Glenn Pollefeyt, Terneuzen (NL); Steven J. Rozeveld, Midland, MI (US); Ewa A. Tocha-Bielak, Terneuzen (NL); Stuart Leadley, Seneffe (BE); Vera Santos, Terneuzen (NL); David F. Yancey, Midland, MI (US); Andrzej Malek, Midland, MI (US)

(73) Assignees: Dow Global Technologies LLC, Midland, MI (US); Dow Silicones Corporation, Midland, MI (US)

(21) Appl. No.: 18/995,486

(22) PCT Filed: Jul. 21, 2023
(86) PCT No.: PCT/US2023/070661

§ 371 Date: Jan. 16, 2025

Related U.S. Application Data
(60) Provisional application No. 63/391,487, filed on Jul. 22, 2022.

(51) Int. Cl. B01J 23/83; B01J 29/85; B01J 35/40; B01J 37/02; B01J 37/12; C07C 1/04

(52) U.S. Cl. CPC B01J 23/83; B01J 29/85; B01J 35/40; B01J 37/0203; B01J 37/12; C07C 1/0435; C07C 2523/63; C07C 2523/83

(57) ABSTRACT

According to embodiments, a method for preparing a self-bound hybrid catalyst may comprise mixing a powder mixture and a binder component to form a hybrid base catalyst and adding an impregnation solution comprising gallium to the hybrid base catalyst to form the self-bound hybrid catalyst after drying and calcination. According to embodiments, a process for preparing C₂ to C₄ hydrocarbons may comprise introducing a feed stream comprising hydrogen gas and a carbon-containing gas selected from the group consisting of carbon monoxide, carbon dioxide, and mixtures thereof into a reaction zone of a reactor and converting the feed stream into a product stream comprising C₂ to C₄ hydrocarbons in the reaction zone in the presence of a self-bound hybrid catalyst formed according to the methods described herein.

### CROSS REFERENCE TO RELATED APPLICATION

This application claims the benefit of U.S. Provisional Patent Application Ser. No. 63/391,487 filed Jul. 22, 2022, which is hereby incorporated by reference in its entirety.

### TECHNICAL FIELD

The present disclosure relates to the preparation of self-bound hybrid catalysts and the application of using the self-bound hybrid catalysts to achieve a high conversion of synthesis gas feeds resulting in good conversion of carbon and high yield of desired products.

### BACKGROUND

For a number of industrial applications, hydrocarbons are used, or are starting materials used, to produce plastics, fuels, and various downstream chemicals. Such hydrocarbons include C₂ to C₄ materials, such as ethene, propene, and butenes (also commonly referred to as ethylene, propylene, and butylenes, respectively). A variety of processes for producing these lower hydrocarbons have been developed, including petroleum cracking and various synthetic processes.

Synthetic processes for converting feed carbon to desired products, such as hydrocarbons, are known. Different types of catalysts have been explored, as well as different kinds of feed streams and proportions of feed stream components. As catalysts typically need to be loaded into commercial reactors in a shaped form, such catalysts generally include an inert and/or inactive binder to provide structural stability.

Consequently, formed hybrid catalysts comprising an inert binder can typically suffer from low carbon conversion when used in these synthetic processes as they dilute the concentration of active components, where much of the feed carbon either (1) does not get converted and exits the process in the same form as the feed carbon; (2) is converted to CO₂; or (3) the synthetic processes have low stability over time and the catalyst rapidly loses its activity for carbon conversion to desirable products. For example, many synthetic processes tend to have reduced COₓ conversion—and, thus, decreased C₂ to C₄ hydrocarbon production-over time. Accordingly, a need exists for catalysts and processes of preparing a catalyst with high on stream stability.

### SUMMARY

It has been found that many conventional catalysts employ an inactive binder that can facilitate the migration of a dopant from the mixed metal oxide catalyst component of a hybrid catalyst to the binder, which can compromise the long-term stability of these catalysts. Further, the inactive binder dilutes the active components in the reactor volume.

Embodiments of the present disclosure address these and other needs by preparation of self-bound hybrid catalysts and processes of using such catalysts. A method of preparing a self-bound hybrid catalyst comprises adding an impregnation solution to a hybrid base catalyst support. The hybrid base catalyst support comprises a metal oxide catalyst support, a microporous catalyst component, and a binder, where the metal oxide catalyst support and the microporous catalyst component are combined into a single catalyst body using the binder. The binder in the hybrid base catalyst support essentially consists of the same material as the metal oxide catalyst support. The impregnation solution comprises a solution comprising one or more dopants that introduces the dopant or dopants onto the hybrid base catalyst support in order to form the self-bound hybrid catalyst. This self-bound hybrid catalyst can then be used for the direct conversion of a feed stream comprising hydrogen gas and a carbon-containing gas, such as syngas, to C₂ to C₄ hydrocarbons. The metal oxide catalyst support and the microporous catalyst component may operate in tandem so that the self-bound hybrid catalyst is able to directly and selectively convert a feed stream comprising hydrogen and carbon-containing gas, such as syngas, to C₂ to C₄ hydrocarbons with high olefin/paraffin ratio.

According to one or more embodiments of the present disclosure, a method for preparing a self-bound hybrid catalyst may comprise mixing a powder mixture and a binder component to form a hybrid base catalyst and adding an impregnation solution comprising gallium to the hybrid base catalyst to form the self-bound hybrid catalyst after drying and calcination. The powder mixture may comprise a metal oxide catalyst support and an 8-membered ring (8-MR) microporous catalyst component, where the metal oxide catalyst support may comprise zirconia. The binder component may comprise a zirconium salt solution, a zirconium salt gel, a zirconium salt slurry, a slurry of carbonates or oxides or hydroxides of zirconium or a colloidal solution of carbonates or oxides or hydroxides of zirconium, where the binder component may have a pH of from 2-8.

According to one or more embodiments of the present disclosure, a process for preparing C₂ to C₄ hydrocarbons may comprise introducing a feed stream comprising hydrogen gas and a carbon-containing gas selected from the group consisting of carbon monoxide, carbon dioxide, and mixtures thereof into a reaction zone of a reactor and converting the feed stream into a product stream comprising C₂ to C₄ hydrocarbons in the reaction zone in the presence of a self-bound hybrid catalyst formed according to the methods of the present disclosure.

Additional features and advantages will be set forth in the detailed description that follows and, in part, will be readily apparent to those skilled in the art from that description or recognized by practicing the embodiments described herein, including the detailed description which follows and the claims.

It is to be understood that both the foregoing general description and the following detailed description describe various embodiments and are intended to provide an overview or framework for understanding the nature and character of the claimed subject matter.

### DETAILED DESCRIPTION

Reference will now be made in detail to embodiments of methods for preparing C₂ to C₄ hydrocarbons and preparing self-bound hybrid catalysts. In one or more embodiments, methods for preparing self-bound hybrid catalysts may comprise mixing a powder mixture and a binder component to form a hybrid base catalyst support and adding an impregnation solution to the hybrid base catalyst support to form the self-bound hybrid catalyst. The powder mixture may comprise a metal oxide catalyst support and a microporous catalyst component. The metal oxide catalyst support may comprise zirconia. The binder component may comprise one or more of a zirconium salt solution, a zirconium salt gel, a zirconium salt slurry, a slurry of zirconium carbonates, oxides, or hydroxides, or a colloidal solution of zirconium carbonates, oxides, or hydroxides. The binder component may have a pH of from 2-8.

In one or more embodiments, methods of preparing C₂ to C₄ hydrocarbons may comprise introducing a feed stream comprising hydrogen gas and a carbon-containing gas selected from a group consisting of carbon monoxide, carbon dioxide, and mixtures thereof into a reaction zone of a reactor and converting the feed stream into a product stream comprising C₂ to C₄ hydrocarbons in the reaction zone in the presence of a self-bound hybrid catalyst. The self-bound hybrid catalyst may be formed by the methods for preparing self-bound hybrid catalysts described herein.

The use of hybrid catalysts is known in the field of hydrocarbon products, such as diesel, or aromatics. However, many known hybrid catalysts are inefficient for forming C₂ to C₄ hydrocarbons, C₂ to C₄ paraffins and C₂ to C₄ olefins, from a feed stream comprising hydrogen gas and a carbon-containing gas because they exhibit a low feed carbon conversion and/or deactivate quickly as they are used. In addition, many known hybrid catalysts employ an inactive binder, often comprising alumina, which can facilitate the migration of a dopant from the metal oxide catalyst support of the hybrid catalyst to the binder and can compromise the long-term stability of the catalyst. Furthermore, without being bound by any particular theory, it is believed that the inactive binder of the hybrid catalyst decreases the amount of active material on the hybrid catalyst that can be used to form C₂ to C₄ hydrocarbons from a feed stream comprising hydrogen gas and a carbon-containing gas, which can cause decreased performance of the catalyst on stream due to an inactive binder taking up volume and diluting the active components in a fixed reactor volume.

In contrast, the self-bound hybrid catalysts disclosed and described herein utilize an active zirconia binder, therefore preventing negative impact from any migration of a dopant or dopants from a metal oxide catalyst support to an inactive binder, thus increasing the long-term stability of the catalyst.

Self-bound hybrid catalysts exhibit a high and steady yield of particularly C₂ to C₄ hydrocarbons-even as the catalyst time on stream increases-when compared to hybrid catalysts where the metal oxide catalyst support and the microporous catalyst component are physically mixed (e.g., are not formed together into a single pellet-bound hybrid catalyst). Without being bound by a theory and as a summary, self-bound hybrid catalysts closely couple independent reactions on each of the two independent catalysts. In the first step, a feed stream comprising hydrogen gas (H₂) and a carbon-containing gas selected from the group consisting of carbon monoxide (CO), carbon dioxide (CO₂), or a mixture of CO and CO₂, such as syngas, may be converted into an intermediate(s) such as oxygenated hydrocarbons, primarily by the metal oxide catalyst component. In the subsequent step, these intermediates may be converted into a product stream comprising hydrocarbons (mostly short chain hydrocarbons, such as, for example, C₂ to C₄ hydrocarbons), primarily by the microporous catalyst component. The continued formation and consumption of the intermediate oxygenates formed in the first step by the reactions of the second step ensures that there is no thermodynamic limit on conversions. In one or more embodiments, a self-bound hybrid catalyst can be formed by mixing powders of the metal oxide catalyst support component and the microporous catalyst component with a binder solution, in which the binder solution comprises the same metal component as found in the metal oxide catalyst support.

In one or more embodiments, the term “metal oxide catalyst support” may refer to a support material of the overall self-bound hybrid catalyst where the support material comprises zirconium oxide (i.e., zirconia). In one or more embodiments, the term “microporous catalyst component” may refer to a molecular sieve zeolitic component comprising crystalline aluminosilicates having a three-dimensional interconnecting network of [SiO₄] and [AlO₄]⁻ tetrahedra. In one or more embodiments, the term “microporous catalyst component” may refer to a molecular sieve zeotype component comprising crystalline silico-aluminophosphates having a three-dimensional interconnecting network of [PO₄]⁺ and [AlO₄]⁻ tetrahedra, in which aluminum or phosphorus are partially replaced by silicon. In one or more embodiments, the term “hybrid base catalyst support” may refer to a composition where the metal oxide catalyst support and microporous catalyst component are bound together by a binder component where the binder component may consist essentially of the same material or materials as the metal oxide catalyst support. In one or more embodiments, the term “self-bound hybrid catalyst” may refer to a catalyst where the hybrid base catalyst support is impregnated with one or more dopants which may comprise, but is not limited to, gallium and lanthanum.

In embodiments, the metal oxide catalyst component is supported on zirconia (ZrO₂). In embodiments, the zirconia support of the metal oxide catalyst component can be phase pure zirconia. As used herein, “phase pure zirconia” refers to zirconia to which no other materials have intentionally been added during formation. Thus, phase pure zirconia includes zirconia with small amounts of components other than zirconium (including oxides other than zirconia) that are unintentionally present in the zirconia as a natural part of the zirconia formation process, such as, for example, hafnium (Hf). In other embodiments, the zirconia can be non-phase pure zirconia, such as zirconia doped with calcium, yttria, lanthanum, cerium or rare earth elements. In embodiments, the zirconia can include zirconia particles having a crystalline structure. In embodiments, the zirconia include zirconia particles having a monoclinic structure. In one or more embodiments, the zirconia consists essentially of or consists of crystalline zirconia particles. In embodiments, the zirconia consist essentially of or consist of monoclinic zirconia particles.

In embodiments, the metal oxide catalyst component comprises gallium oxide. As used herein, “gallium oxide” refers to gallium in various oxidation states. In embodiments, gallium oxide can be deposited on the surface of zirconia or be in solid solution with ZrO₂. In other embodiments, gallium oxide may include but not be limited to Ga₂O₃, GaO(OH), and Ga₅O₇(OH). Gallium oxide can also include polymorphs of Ga₂O₃, such as monoclinic (β-Ga₂O₃), rhombohedral (α-Ga₂O₃), defective spinel (γ-Ga₂O₃), cubic (δ-Ga₂O₃), or orthorhombic (ε-Ga₂O₃) structures. In other embodiments, gallium oxide may include gallium in more than one oxidation state. For example, individual gallium may be in different oxidation states. Gallium oxide is not limited to comprising gallium in homogenous oxidation states.

In one or more embodiments, the metal oxide catalyst support may be mixed with a microporous catalyst component. The microporous catalyst component may be, in embodiments, selected from molecular sieves having 8-MR (member ring) pore openings and having a framework type selected from the group consisting of the following framework types: CHA, AEI, AFX, ERI, LEV, LTA, UFI, RTH, EDI, GIS, MER, RHO, and combinations thereof, the framework types corresponding to the naming convention of the International Zeolite Association. It should be understood that in embodiments, both aluminosilicate and silicoaluminophosphate frameworks may be used. Embodiments may include tetrahedral aluminosilicates, ALPOs (such as, for example, tetrahedral aluminophosphates), SAPOs (such as, for example, tetrahedral silicoaluminophosphates), and silica-only based tectosilicates. In certain embodiments, the microporous catalyst component may be silicoaluminophosphate having a Chabazite (CHA) framework type. Examples of these may include, but are not necessarily limited to: CHA embodiments selected from SAPO-34 and SSZ-13 and AEI embodiments such as SAPO-18 and SSZ-39. Combinations of microporous catalyst components having any of the above framework types may also be employed. It should be understood that the microporous catalyst component may have different membered ring pore opening depending on the desired product. For instance, a microporous catalyst component having 8-MR to 12-MR pore openings could be used depending on the desired product. However, to produce C₂ to C₄ hydrocarbons, a microporous catalyst component having 8-MR pore openings is used in embodiments.

In one or more embodiments, a binder solution may be mixed with the powder mixture of the metal oxide catalyst support and the microporous catalyst component to form the hybrid base catalyst support. The binder solution may comprise a zirconium salt solution, a zirconium salt gel, a zirconium salt slurry, a slurry of zirconium carbonates, oxides, or hydroxides, or a colloidal solution of zirconium carbonates, oxides, or hydroxides. In one embodiment, the binder solution may consist of zirconium acetate. In another embodiment, the binder solution may consist of zirconyl nitrate. In another embodiment, the binder solution may consist of zirconium oxide or zirconium hydroxide. In embodiments, the binder solution may comprise a mixture of one or more of zirconium acetate, zirconyl nitrate, ammonium zirconium carbonate, zirconium hydroxide and zirconium oxide.

In one or more embodiments, the binder solution may have a pH of from 2.0 to 8.0, such as from 2.5 to 8.0, from 3.0 to 8.0, from 3.5 to 8.0, from 4.0 to 8.0, from 4.5 to 8.0, from 5.0 to 8.0, from 5.5 to 8.0, from 2.0 to 5.5, from 2.5 to 5.5, from 3.0 to 5.5, from 3.5 to 5.5, from 4.0 to 5.5, from 4.5 to 5.5, from 5.0 to 5.5, from 2.0 to 5.0, from 2.5 to 5.0, from 3.0 to 5.0, from 3.5 to 5.0, from 4.0 to 5.0, from 4.5 to 5.0, from 2.0 to 4.5, from 2.5 to 4.5, from 3.0 to 4.5, from 3.5 to 4.5, from 4.0 to 4.5, from 2.0 to 4.0, from 2.5 to 4.0, from 3.0 to 4.0, from 3.5 to 4.0, from 2.0 to 3.5, from 2.5 to 3.5, from 3.0 to 3.5, from 2.0 to 3.0, from 2.5 to 3.0, or from 2.0 to 2.5. For example, in embodiments, the pH of the binder solution may be 2, 2.5, 3, 3.5, 4, 4.5, 5, 5.5, 6, 6.5, 7, 7.5, or 8. Without being bound by a theory, it is believed that when the binder solution has a pH over 8, the binder solution becomes too basic and may compromise the structural integrity of the microporous catalyst component when the binder solution is added to the powder mixture. Further, without being bound by a theory, it is believed that when the binder solution has a pH lower than 2, the binder solution becomes too acidic and may compromise the structural integrity of the microporous catalyst component when the binder solution is added to the powder mixture.

The metal oxide catalyst support, the microporous catalyst component, and the binder solution may be mixed together by any suitable means to achieve homogenous mixing of all the components prior to extrusion. The metal oxide catalyst support and the microporous catalyst component can be initially mixed as powders to achieve homogeneity in a suitable dry mixer, such as a ribbon or plow mixer. The metal oxide catalyst support, the microporous catalyst component, and the binder solution may be combined to result in a catalyst having a mass ratio of from 1:10 to 10:1, such that the mass ratio of metal oxide catalyst support and binder component to microporous catalyst component is 1:1, 1:2, 1:3, 1:4, 1:5, 1:6, 1:7, 1:8, 1:9, or 1:10 and the mass ratio of microporous catalyst component to metal oxide catalyst support and binder component is 1:1, 1:2, 1:3, 1:4, 1:5, 1:6, 1:7, 1:8, 1:9, or 1:10.

After mixing the powder mixture and the binder solution, the hybrid base catalyst support may be formed. In embodiments, mixing the powder mixture and the binder solution may form an initial paste. The initial paste may be mixed until an extrudable paste is formed and the hybrid base catalyst support is formed using means known to one skilled in the art. The hybrid base catalyst support may then be dried at temperatures of at least 40° C. For example, the hybrid base catalyst support may be dried at a temperature of 40° C. to 200° C. In embodiments, the hybrid base catalyst support may be dried at a temperature from 60° C. to 200° C., from 80° C. to 200° C., from 100° C. to 200° C., from 120° C. to 200° C., from 140° C. to 200° C., or from 160° C. to 200° C. In embodiments, the hybrid base catalyst support may be dried at a temperature of from 40° C. to 180° C., from 40° C. to 160° C., from 40° C. to 140° C., from 40° C. to 120° C., from 40° C. to 100° C., or from 40° C. to 80° C. In embodiments, the hybrid base catalyst support is further used in its dried form. In alternative embodiments, the dried hybrid base catalyst support may be calcined at temperatures of from 200° C. to 800° C. in order to form the hybrid base catalyst support. In embodiments, the dried hybrid base catalyst support may be calcined at temperatures of from 200° C. to 700° C., 200° C. to 600° C., 200° C. to 500° C., 200° C. to 400° C., or 200° C. to 300° C. In embodiments, the dried extrudable paste may be calcined at temperatures of from 300° C. to 800° C., from 400° C. to 800° C., from 500° C. to 800° C., from 600° C. to 800° C., or from 700° C. to 800° C.

In embodiments, the hybrid base catalyst support may have a particle size from 0.5 mm to 6.0 mm. For example, the hybrid base catalyst support may have a particle size of at least 0.5 mm, at least 1.0 mm, at least 1.5 mm, at least 2.0 mm, at least 2.5 mm, at least 3.0 mm, at least 3.5 mm, at least 4.0 mm, at least 4.5 mm, at least 5.0 mm, at least 5.5 mm, or even 6.0 mm. In embodiments, the hybrid base catalyst support may have a particle size ranging from 0.5 mm to 5.5 mm, from 0.5 mm to 5.0 mm, from 0.5 mm to 4.5 mm, from 0.5 mm to 4.0 mm, from 0.5 mm to 3.5 mm, from 0.5 mm to 3.0 mm, from 0.5 mm to 2.5 mm, from 0.5 mm to 2.0 mm, or from 0.5 mm to 1.5 mm. In embodiments, the hybrid base catalyst support may have a particle size ranging from 1.0 mm to 6.0 mm, from 1.5 mm to 6.0 mm, from 2.0 mm to 6.0 mm, from 2.5 mm to 6.0 mm, from 3.0 mm to 6.0 mm, from 3.5 mm to 6.0 mm, from 4.0 mm to 6.0 mm, from 4.5 mm to 6.0 mm, from 5.0 mm to 6.0 mm, or from 5.5 mm to 6.0 mm. The particle size may be essentially the shortest dimension of the catalyst particle. For example, when the hybrid base catalyst support has a hollow cylinder or a ring shape, the particle size is the thickness of the hollow cylinder wall. When the hybrid base catalyst support has a spherical shape, the particle size is the diameter of the sphere. The particle size of the hybrid base catalyst support may be controlled by choice of the extrusion die diameter and measured by dynamic image analysis methods.

In one or more embodiments, an impregnation solution may be added to the hybrid base catalyst support in order to form the self-bound hybrid catalyst. In one or more embodiments, the impregnation solution may comprise lanthanum. In one or more embodiments, the impregnation solution may comprise one or more rare earth elements. The rare earth elements may include scandium, yttrium, and elements having an atomic number from 57 to 71, such as, but not limited to, samarium, gadolinium, dysprosium, cerium, and neodymium. In embodiments, the impregnation solution may comprise gallium (III) nitrate (i.e., Ga(NO₃)₃). In embodiments, the impregnation solution may comprise lanthanum (III) nitrate (i.e., La(NO₃)₃). In embodiments, the impregnation solution may comprise both gallium (III) nitrate and lanthanum (III) nitrate. The gallium may, in embodiments be added via a gallium-containing precursor.

In one or more embodiments, the impregnation solution may comprise transition metal elements such as nickel (Ni), palladium (Pd), or platinum (Pt). In embodiments, the impregnation solution comprises a base and may have a pH of less than or equal to 10.0. For example, the impregnation solution may have a pH of from 2.0 to 10.0. In embodiments, the impregnation solution may have a pH of from 2.5 to 7.5, from 3.0 to 7.5, from 3.5 to 7.5, from 4.0 to 7.5, or from 4.5 to 7.5. In embodiments, the impregnation solution may have a pH of from 2.0 to 4.5, from 2.0 to 4.0, from 2.0 to 3.5, from 2.0 to 3.0, or from 2.0 to 2.5. In embodiments, the impregnation solution may have a pH of from 5.0 to 10.0, from 5.5 to 10.0, from 6.0 to 10.0, from 6.5 to 10.0, from 7.0 to 10.0, from 7.5 to 10.0, or from 8.0 to 10.0.

In embodiments, the impregnation solution may comprise one or more chelating or complexing agents to ensure solubility of the metal ions at desired concentrations and at a desired pH. The term “chelating agent” or “complexing agent” refers to a chemical agent that can help facilitate the stabilization of metal ion solution via bonding to said metal ion and forming a soluble complex. In some embodiments, the chelating agent and/or complexing agent may comprise carboxylic acids, iminoacids, amines, glycols, or combinations thereof. In some embodiments, the impregnation solution may comprise ammonia, ethanolamine, diethanolamine, triethanolamine, or combinations thereof in place of or in addition to the chelating agent and/or complexing agent.

After mixing the hybrid base catalyst support and the impregnation solution, self-bound hybrid catalyst may be formed. The self-bound hybrid catalyst may be dried at temperatures of at least 40° C. For example, the self-bound hybrid catalyst may be dried at a temperature of 40° C. to 200° C. In embodiments, self-bound hybrid catalyst may be dried at a temperature from 60° C. to 200° C., from 80° C. to 200° C., from 100° C. to 200° C., from 120° C. to 200° C., from 140° C. to 200° C., or from 160° C. to 200° C. In embodiments, the self-bound hybrid catalyst may be dried at a temperature of from 40° C. to 180° C., from 40° C. to 160° C., from 40° C. to 140° C., from 40° C. to 120° C., from 40° C. to 100° C., or from 40° C. to 80° C. Then, the dried self-bound hybrid catalyst may be calcined at temperatures of from 200° C. to 800° C. In embodiments, the dried self-bound hybrid catalyst may be calcined at temperatures of from 200° C. to 700° C., 200° C. to 600° C., 200° C. to 500° C., 200° C. to 400° C., or 200° C. to 300° C. In embodiments, the dried self-bound hybrid catalyst may be calcined at temperatures of from 300° C. to 800° C., from 400° C. to 800° C., from 500° C. to 800° C., from 600° C. to 800° C., or from 700° C. to 800° C.

In embodiments, the self-bound hybrid catalyst may have a particle size from 0.5 mm to 6.0 mm. For example, the self-bound hybrid catalyst may have a particle size of at least 0.5 mm, at least 1.0 mm, at least 1.5 mm, at least 2.0 mm, at least 2.5 mm, at least 3.0 mm, at least 3.5 mm, at least 4.0 mm, at least 4.5 mm, at least 5.0 mm, at least 5.5 mm, or even 6.0 mm. In embodiments, the self-bound hybrid catalyst may have a particle size ranging from 0.5 mm to 5.5 mm, from 0.5 mm to 5.0 mm, from 0.5 mm to 4.5 mm, from 0.5 mm to 4.0 mm, from 0.5 mm to 3.5 mm, from 0.5 mm to 3.0 mm, from 0.5 mm to 2.5 mm, from 0.5 mm to 2.0 mm, or from 0.5 mm to 1.5 mm. In embodiments, the self-bound hybrid catalyst may have a particle size ranging from 1.0 mm to 6.0 mm, from 1.5 mm to 6.0 mm, from 2.0 mm to 6.0 mm, from 2.5 mm to 6.0 mm, from 3.0 mm to 6.0 mm, from 3.5 mm to 6.0 mm, from 4.0 mm to 6.0 mm, from 4.5 mm to 6.0 mm, from 5.0 mm to 6.0 mm, or from 5.5 mm to 6.0 mm. The particle size may be essentially the shortest dimension of the catalyst particle. For example, when the self-bound hybrid catalyst has a hollow cylinder or a ring shape, the particle size is the thickness of the hollow cylinder wall. When the self-bound hybrid catalyst has a spherical shape, the particle size is the diameter of the sphere. The particle size of the self-bound hybrid catalyst may be controlled by choice of the extrusion die diameter and measured by dynamic image analysis methods.

Without being bound by a theory, it is believed that because the impregnation solution is added to the hybrid base catalyst support after the hybrid base catalyst support is formed, dried, and/or calcined, this allows for the contents of the impregnation solution to be added onto the entirety of the hybrid base catalyst support, including the zirconia-based binder of the hybrid base catalyst support. Thus, with the impregnation solution being added to the entirety of the hybrid base catalyst support, including the active zirconia-based binder, the increased amount of active components on the self-bound hybrid catalyst results in an improved yield of C₂ to C₄ hydrocarbons from a feed stream comprising hydrogen gas and a carbon-containing gas.

The self-bound hybrid catalyst may be used in methods for converting carbon in a carbon-containing feed stream to C₂ to C₄ hydrocarbons. As used herein, a “carbon-containing feed stream” may refer to a gas selected from carbon monoxide, carbon dioxide, and mixtures thereof. As used herein, “synthesis gas” or “syngas” may refer to a gas comprising hydrogen gas and a carbon-containing gas. Such processes will be described in more detail below.

According to embodiments, a feed stream may be fed into a reaction zone, the feed stream comprising hydrogen (H₂) gas and a carbon-containing gas selected from carbon monoxide (CO), carbon dioxide (CO₂), and combinations thereof. In embodiments, the H₂ gas is present in the feed stream in an amount of from 10 volume percent (vol. %) to 90 vol. %, based on combined volumes of the H₂ gas and the gas selected from CO, CO₂, and combinations thereof. The feed stream may be contacted with the self-bound hybrid catalyst as disclosed and described herein while in the reaction zone.

Without being bound by a theory, it should be understood that the activity of the self-bound hybrid catalyst will be higher for feed streams containing CO as the carbon-containing gas, and that the activity of the self-bound hybrid catalyst may decrease as a larger portion of the carbon-containing gas in the feed stream is CO₂. However, that is not to say that the self-bound hybrid catalyst disclosed and described herein cannot be used in methods where the feed stream includes CO₂ as all, or a large portion, of the carbon-containing gas.

The feed stream may be contacted with the self-bound hybrid catalyst while in the reaction zone under reaction conditions sufficient to form a product stream comprising C₂ to C₄ hydrocarbons. The reaction conditions may include a temperature within the reaction zone ranging, according to one or more embodiments, from 350° C. to 480° C., such that the temperature is at least 350° C., at least 370° C., at least 390° C., at least 410° C., at least 430° C., or at least 450° C. In embodiments, the temperature within the reaction zone may be from 370° C. to 460° C., from 390° C. to 460° C., or from 390° C. to 450° C. In embodiments, the temperature within the reaction zone may be from 370° C. to 480° C., from 390° C. to 480° C., from 410° C. to 480° C., or from 430° C. to 480° C. In embodiments, the temperature within the reaction zone may be from 350° C. to 460° C., from 350° C. to 440° C., from 350° C. to 420° C., from 350° C. to 400° C., or from 350° C. to 380° C.

In embodiments, the reaction conditions include a pressure inside the reaction zone of at least 1 bar (100 kilopascals (kPa), such as at least 5 bar (500 kPa), at least 10 bar (1,000 kPa), at least 15 bar (1,500 kPa), at least 20 bar (2,000 kPa), at least 25 bar (2,500 kPa), at least 30 bar (3,000 kPa), at least 35 bar (3,500 kPa), at least 40 bar (4,000 kPa), at least 45 bar (4,500 kPa), at least 50 bar (5,000 kPa), at least 55 bar (5,500 kPa), at least 60 bar (6,000 kPa), at least 65 bar (6,500 kPa), at least 70 bar (7,000 kPa), at least 75 bar (7,500 kPa), at least 80 bar (8,000 kPa), at least 85 bar (8,500 kPa), at least 90 bar (9,000 kPa), at least 95 bar (9,500 kPa), or at least 100 bar (10,000 kPa). In other embodiments, the reaction conditions include a pressure inside the reaction zone is from 5 bar (500 kPa) to 100 bar (10,000 kPa), such as from 10 bar (1,000 kPa) to 95 bar (9,500 kPa), from 15 bar (1,500 kPa) to 90 bar (9,000 kPa), from 20 bar (2,000 kPa) to 85 bar (8,500 kPa), from 25 bar (2,500 kPa) to 80 bar (8,000 kPa), from 30 bar (3,000 kPa) to 75 bar (7,500 kPa), from 35 bar (3,500 kPa) to 70 bar (7,000 kPa), from 40 bar (4,000 kPa) to 65 bar (6,500 kPa), from 45 bar (4,500 kPa) to 60 bar (6,000 kPa), or from 50 bar (5,000 kPa) to 55 bar (5,500 kPa). In embodiments, the pressure inside the reaction zone is from 20 bar (2,000 kPa) to 60 bar (6,000 kPa).

According to embodiments, the gas hourly space velocity (GHSV) within the reaction zone is from 500 per hour (/h) to 12,000/h, such as from 500/h to 10,000/h, from 1,200/h to 12,000/h, from 1,500/h to 10,000/h, from 2,000/h to 9,500/h, from 2,500/h to 9,000/h, from 3,000/h to 8,500/h, from 3,500/h to 8,000/h, from 4,000/h to 7,500/h, from 4,500/h to 7,000/h, from 5,000/h to 6,500/h, or from 5,500/h to 6,000/h. In embodiments the GHSV within the reaction zone is from 1,800/h to 3,600/h, such as from 2,000/h to 3,600/h, from 2,200/h to 3,600/h, from 2,400/h to 3,600/h, from 2,600/h to 3,600/h, from 2,800/h to 3,600/h, from 3,000/h to 3,600/h, from 3,200/h to 3,600/h, or from 3,400/h to 3,600/h. In embodiments, the GHSV within the reaction zone is from 1,800/h to 3,400/h, such as from 1,800/h to 3,200/h, from 1,800/h to 3,000/h, from 1,800/h to 2,800/h, from 1,800/h to 2,600/h, from 1,800/h to 2,400/h, from 1,800/h to 2,200/h, or from 1,800/h to 2,000/h. In embodiments, the GHSV within the reaction is from 2,000/h to 3,400/h, such as from 2,200/h to 3,200/h, from 2,400/h to 3,000/h, or from 2,600/h to 2,800/h.

In embodiments, when using the self-bound hybrid catalysts disclosed and described herein along with the process conditions disclosed and described herein, the carbon conversion may be improved. Within the process ranges disclosed, the conversion of the feed containing carbon oxides and hydrogen can be carried out in a series of rectors with an intermediate knock-out of water by-product by the means of e.g., phase separation, membrane separation, or some type of water-selective absorptive or adsorptive process. Further, without being bound by a theory, directing the partially converted and water-free effluent to the subsequent reactor in series and repeating this manner of technological operations will have an overall effect of enhancing the hydrocarbon yield.

### EXAMPLES

The following examples illustrate features of the present disclosure, but are not intended to limit the scope of the disclosure. For each of the following examples and comparative examples, the microporous catalyst component was prepared as follows: SAPO-34 was synthesized per literature procedures (Lok, B. M.; Messina, C. A.; Patton, R. L.; Gajek, R. T.; Cannan, T. R.; Flanigen, E. M. Crystalline silicoaluminophosphates. U.S. Pat. No. 4,440,871A, 1984). When using calcined SAPO-34, the materials were calcined in air using the following program: 25° C. raise to 600° C. at a heating rate of 2° C./min, hold at 600° C. for 4 hours (h), cool down to 25° C. in 4 h. The material was sieved to a fraction smaller than 200 mesh (smaller than 75 m).

### Example 1

A hybrid base catalyst support was prepared by mixing 4 grams of the zirconium oxide component (manufactured by Daiichi Kigenso Kagaku-Kogyo Co., Ltd with the tradename RC-100) with 1 gram of uncalcined SAPO-34 (<200 mesh size (75 μm)) for 2 minutes using a mortar and pestle. Separately, 15 mL of a 20 wt. % zirconium acetate solution (manufactured by Daiichi Kigenso Kagaku-Kogyo Co., Ltd with the tradename Zircosol ZA-20) was partially neutralized by slowly adding a 20 wt. % ammonium zirconium carbonate (AZC) solution (manufactured by Daiichi Kigenso Kagaku-Kogyo Co., Ltd with the tradename Zircosol AC-20) at approximately 50° C. under stirring until a pH of 5-6 was obtained (addition of a total of 1.8 mL AZC solution). The Zr-containing binder solution was added to the dry mixed powders, targeting a final ZrO₂ binder concentration of 20 wt. % on total solid basis. The paste was subsequently mixed for at least 15 minutes using a mortar and pestle until an extrudable paste was obtained. The paste was transferred to a ceramic dish and dried at 85° C. overnight. The dried precursor was heated in a static muffle furnace at 2° C./min to 250° C. and held at that temperature for 4 hours.

After calcination, the hybrid base catalyst support was crushed and sieved to 40-80 mesh.

A stock solution for impregnation was prepared. La(NO₃)₃×6 H₂O (0.9752 grams) and Ga(NO₃)₃ 2.04 M stock solution (3.6805 mL) were added into a beaker and stirred. Citric acid (1.875 grams) was added and stirred until dissolved. The solution was slowly neutralized until a pH of 5 was achieved using concentrated ammonia. Water was added to a total volume of 10 mL and homogenized.

The 40-80 mesh fraction was impregnated with the Ga and La dopants by slowly adding the stock solution to the meshed base catalyst in a vortex mixer solution (approximately 0.44 mL per gram of base catalyst support). The impregnated meshed fraction was dried at 85° C. overnight. The dried self-bound hybrid catalyst was then heated in a static muffle furnace at 2° C./min to 600° C. and held at that temperature for 4 hours. After calcination, the self-bound hybrid catalyst was sieved to 40-80 mesh for testing.

### Example 2

A hybrid base catalyst support was prepared by mixing 4 grams of the zirconium oxide component (manufactured by Daiichi Kigenso Kagaku-Kogyo Co., Ltd with the tradename Z3186) with 1 gram of uncalcined SAPO-34 (<200 mesh size (75 μm)) for 2 minutes using a mortar and pestle. Separately, 15 mL of a 20 wt. % zirconium acetate solution (manufactured by Daiichi Kigenso Kagaku-Kogyo Co., Ltd with the tradename Zircosol ZA-20) was partially neutralized by slowly adding a 20 wt. % ammonium zirconium carbonate (AZC) solution (manufactured by Daiichi Kigenso Kagaku-Kogyo Co., Ltd with the tradename Zircosol AC-20) at approximately 50° C. under stirring until a pH of 5-6 was obtained (addition of a total of 1.8 mL AZC solution). The Zr-containing binder solution was added to the dry mixed powders, targeting a final ZrO₂ binder concentration of 20 wt. % on total solid basis. The paste was subsequently mixed for at least 15 minutes using a mortar and pestle until an extrudable paste was obtained. The paste was transferred to a ceramic dish and dried at 85° C. overnight. The dried precursor was heated in a static muffle furnace at 2° C./min to 250° C. and held at that temperature for 4 hours.

After calcination, the hybrid base catalyst support was crushed and sieved to 40-80 mesh.

A stock solution for impregnation was prepared. La(NO₃)₃×6 H₂O (0.9752 grams) and Ga(NO₃)₃ 2.04 M stock solution (3.6805 mL) were added into a beaker and stirred. Citric acid (1.875 grams) was added and stirred until dissolved. The solution was slowly neutralized until a pH of 5 was achieved using concentrated ammonia. Water was added to a total volume of 10 mL and homogenized.

The 40-80 mesh fraction was impregnated with the Ga and La dopants by slowly adding the stock solution to the meshed base catalyst in a vortex mixer solution (approximately 0.44 mL per gram of base catalyst support). The impregnated meshed fraction was dried at 85° C. overnight. The dried self-bound hybrid catalyst was then heated in a static muffle furnace at 2° C./min to 600° C. and held at that temperature for 4 hours. After calcination, the self-bound hybrid catalyst was sieved to 40-80 mesh for testing.

### Comparative Example 1

A metal oxide catalyst component comprising gallium on a zirconia support was prepared by an incipient wetness impregnation method. An impregnation solution of Ga(NO₃)₃×H₂O and La(NO₃)₃×6 H₂O with a concentration of respectively 0.57 mol/L and 0.17 mol/L in deionized water was prepared. 10 grams of ZrO₂ support (manufactured by Daiichi Kigenso Kagaku-Kogyo Co., Ltd with the tradename RC-100, >94% monoclinic phase by XRD, pore volume=0.7 mL/g measured by deionized water) was weighed and placed into a glass vial. Subsequently, 7 mL of the Ga/La impregnation solution was added dropwise to the support while constantly shaking. After impregnation, the catalyst was dried at 85° C. in the oven overnight and subsequently heated with 3° C./min to 550° C. and kept at that temperature for 4 hours. After calcination, the catalyst was sieved to <200 mesh (75 m) to remove larger agglomerated particles.

A hybrid catalyst was prepared by mixing 8 grams of the metal oxide catalyst component described above with 1.808 grams of uncalcined SAPO-34 (<200 mesh size (75 m)) for 10 minutes using a mortar and pestle. Separately, pseudoboehmite (AlOOH) (manufactured by Sasol Limited, tradename Catapal D) was peptized in water using HNO₃ (65 wt. % in H₂O) at a [HNO₃]/[Al] ratio of 0.05, and a total solid content of 35 wt. %. The peptized pseudoboehmite mixture was added to the dry mixed powders, targeting a pseudoboehmite concentration of 24.6 wt. % on total solids basis. The paste was subsequently mixed for at least 10 minutes using a mortar and pestle until an extrudable paste was obtained. The paste was transferred to a ceramic dish and dried at 85° C. overnight. The dried precursor was heated in a static muffle furnace at 2° C./min to 600° C. and held at that temperature for 4 hours. After calcination, the hybrid catalyst was crushed and sieved to 40-80 mesh for testing.

### Comparative Example 2

A metal oxide catalyst component comprising gallium on a zirconia support was prepared by an incipient wetness impregnation method. An impregnation solution of Ga(NO₃)₃×H₂O and La(NO₃)₃×6 H₂O with a concentration of respectively 1 mol/L and 0.3 mol/L in deionized water was prepared. 10 grams of ZrO₂ support (manufactured by Daiichi Kigenso Kagaku-Kogyo Co., Ltd with the tradename Z3186, >94% monoclinic phase by XRD, pore volume=0.55 mL/g measured by deionized water) was weighed and placed into a glass vial. After that, 5.5 mL of the Ga/La impregnation solution was added dropwise to the support while constantly shaking. After impregnation, the catalyst was dried at 85° C. in the oven overnight and subsequently heated at 3° C./min to 550° C. and kept at that temperature for 4 hours. After calcination, the catalyst was sieved to <200 mesh (75 m) to remove larger agglomerated particles.

A hybrid catalyst was prepared by mixing 8 grams of the metal oxide catalyst component described above with 1.808 grams of uncalcined SAPO-34 (<200 mesh size (75 m)) for 10 minutes using a mortar and pestle. Separately, pseudoboehmite (AlOOH) (manufactured by Sasol Limited, tradename Catapal D) was peptized in water using HNO₃ (65 wt. % in H₂O) at a [HNO₃]/[Al] ratio of 0.05, and a total solid content of 35 wt. %. The peptized pseudoboehmite mixture was added to the dry mixed powders, targeting a pseudoboehmite concentration of 24.6 wt. % on total solids basis. The paste was subsequently mixed for at least 10 minutes using a mortar and pestle until an extrudable paste was obtained. The paste was transferred to a ceramic dish and dried at 85° C. overnight. The dried precursor was heated in a static muffle furnace at 2° C./min to 600° C. and held at that temperature for 4 hours. After calcination, the hybrid catalyst was crushed and sieved to 40-80 mesh for testing.

### Comparative Example 3

A hybrid base catalyst support was prepared by mixing 4 grams of zirconium oxide (manufactured by Daiichi Kigenso Kagaku-Kogyo Co., Ltd with the tradename RC-100) with 1 gram of uncalcined SAPO-34 (<200 mesh size (75 μm)) for 2 minutes using a mortar and pestle. A 20 wt. % ammonium zirconium carbonate binder solution (manufactured by Daiichi Kigenso Kagaku-Kogyo Co., Ltd with the tradename Zircosol ZA-20, pH≈10) was added to the dry mixed powders, targeting a final ZrO₂ binder concentration of 20 wt. % on total solid basis. The paste was subsequently mixed for at least 15 minutes using a mortar and pestle until an extrudable paste was obtained. The paste was transferred to a ceramic dish and dried at 85° C. overnight. The dried precursor was heated in a static muffle furnace at 2° C./min to 600° C. and held at that temperature for 4 hours.

It was noticed that the basic ammonium zirconium carbonate solution (pH≈10) damages the structural integrity of SAPO-34, which is detrimental for the productivity of the catalyst. The expected micropore volume of the hybrid catalyst was reduced to ˜80% as compared to >95% for inventive Examples 1 and 2, where the pH was about 5-6. Thus, the pH of the zirconia binder solution is of vital importance to reduce damage to the SAPO-34. Therefore, we did not perform reactor testing for Comparative Example 3 in a reactor.

### Catalytic Performance Data

Testing of the hybrid catalysts was performed in a stainless steel fixed bed reactor system (7.7 mm internal diameter) under the following conditions: 420° C., H₂/CO=3, pressure=40 bar, weight hourly space velocity (WHSV)=1.54 hr⁻¹.

Prior to contacting with syngas, the self-bound catalyst was heated under nitrogen (N₂) to reaction temperature and pressure. The reactor effluent composition was obtained by gas chromatography and the conversion and carbon based selectivities were calculated using the following equations:

$${X}_{CO} ( \% ) = [ ( {η}_{CO , in} - {η}_{CO , out} ) / {η}_{CO , in} ] \cdot 100 ; and \qquad ( 1 )$$

$${S}_{j} ( \% ) = [ {α}_{i} \cdot {η}_{j , out} / ( {η}_{CO , in} - {η}_{CO , out} ) ] \cdot 100. \qquad ( 2 )$$

where X_{CO} is defined as the CO conversion (%), η_{CO, in} is defined as the molar inlet flow of CO (μmol/s), η_{CO, out} is the molar outlet flow of CO (μmol/s), Sⱼ is defined as the carbon-based selectivity to product j (%), αⱼ is the number of carbon atoms for product j, and η_{j, out} is the molar outlet flow of product j (μmol/s).

The results of the catalytic testing are shown in Table 1 below. The reported conversion values (X_{CO}) are the averaged conversion levels between 50 hours and 120 hours of the time on stream (TOS) of the self-bound catalyst contacting the syngas. The normalized deactivation rate k is calculated by fitting the data between 24 hours and 150 hours to a logarithmic decay: X/X₀=a+k*ln(TOS(h)).

TABLE 1

| | | | C₂-C₅ | C₂-C₄ | |
|---|---|---|---|---|---|
| | | | Paraffin | Olefin Sel. | |
| | X_{CO} (%) | k | Sel. (%) | (%) | O/P |
| Example 1 | 50.7 | −0.047 | 10.6 | 50.3 | 4.7 |
| Example 2 | 58.1 | −0.019 | 13.6 | 46.5 | 3.4 |
| Comparative | 49.4 | −0.061 | 10.8 | 46.7 | 4.3 |
| Example 1 | | | | | |
| Comparative | 59.4 | −0.026 | 9.1 | 51.9 | 5.7 |
| Example 2 | | | | | |
| Comparative | — | — | — | — | — |
| Example 3 | | | | | |

As can be observed in Table 1, zirconia-bound bifunctional catalyst formulations showed a lower deactivation rate (in terms of absolute value) compared to corresponding alumina-bound systems. This behavior was observed for different zirconia grades. For the zirconia grade RC-100, the zirconia-bound inventive Example 1 had a 23% lower deactivation rate compared to the alumina-bound Comparative Example 1. For the zirconia grade Z3186, the zirconia-bound inventive Example 2 had a 27% lower deactivation rate compared to the alumina-bound Comparative Example 2.

The present disclosure includes one or more non-limiting aspects. A first aspect includes a method for preparing a self-bound hybrid catalyst, the method comprising: mixing a powder mixture and a binder component to form a hybrid base catalyst; and adding an impregnation solution comprising gallium to the hybrid base catalyst to form the self-bound hybrid catalyst after drying and calcination, wherein the powder mixture comprises a metal oxide catalyst support and an 8-membered ring (8-MR) microporous catalyst component, the metal oxide catalyst support comprises zirconia, and the binder component comprises a zirconium salt solution, a zirconium salt gel, a zirconium salt slurry, a slurry of carbonates or oxides or hydroxides of zirconium or a colloidal solution of carbonates or oxides or hydroxides of zirconium, where the binder component has a pH of from 2-8.

A second aspect includes any above aspect, wherein the gallium is introduced into the impregnation solution via a gallium-containing precursor.

A third aspect includes any above aspect, wherein the impregnation solution comprises at least one rare earth element.

A fourth aspect includes any above aspect, wherein the impregnation solution comprises one or more of nickel, palladium, or platinum.

A fifth aspect includes any above aspect, the impregnation solution has a pH that is from 2 to 10.

A sixth aspect includes any above aspect, wherein the impregnation solution has a pH that is from 4 to 7.

A seventh aspect includes any above aspect, wherein the impregnation solution comprises a chelating or a complexing agent.

An eighth aspect includes any above aspect, wherein the chelating or a complexing agent is selected from the group consisting of carboxylic acids, iminoacids, amines, glycols, and mixtures thereof.

A ninth aspect includes any above aspect, wherein the impregnation solution comprises citric acid.

A tenth aspect includes any above aspect, wherein binder component comprises at least one of ammonium zirconium carbonate, zirconyl acetate, and zirconium hydroxide.

An eleventh aspect includes any above aspect, wherein the self-bound hybrid catalyst has a particle size that is from 0.5 mm to 6.0 mm.

A twelfth aspect includes any above aspect, wherein the self-bound hybrid catalyst, has a particle size of less than 3.0 mm.

A thirteenth aspect includes any above aspect, wherein the microporous catalyst component comprises SAPO-34.

A fourteenth aspect includes any above aspect, wherein the microporous catalyst component comprises uncalcined SAPO-34.

A fifteenth aspect includes a process for preparing C₂ to C₄ hydrocarbons comprising: introducing a feed stream comprising hydrogen gas and a carbon-containing gas selected from the group consisting of carbon monoxide, carbon dioxide, and mixtures thereof into a reaction zone of a reactor; and converting the feed stream into a product stream comprising C₂ to C₄ hydrocarbons in the reaction zone in the presence of a self-bound hybrid catalyst formed according to the method of any one of the above aspects.

The subject matter of the present disclosure has been described in detail and by reference to specific embodiments. It should be understood that any detailed description of a component or feature of an embodiment does not necessarily imply that the component or feature is essential to the particular embodiment or to any other embodiment. Further, it should be apparent to those skilled in the art that various modifications and variations can be made to the described embodiments without departing from the spirit and scope of the claimed subject matter.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” a second component, it is contemplated that, in embodiments, the first component “consists” or “consists essentially of” that second component. It should further be understood that where a first component is described as “comprising” a second component, it is contemplated that, in embodiments, the first component comprises at least 10%, at least 20%, at least 30%, at least 40%, at least 50%, at least 60%, at least 70%, at least 80%, at least 90%, at least 95%, or even at least 99% of that second component (where % can be weight % or molar %).

For the purposes of describing and defining the present inventive technology, it is noted that reference herein to a variable being a “function” of a parameter or another variable is not intended to denote that the variable is exclusively a function of the listed parameter or variable. Rather, reference herein to a variable that is a “function” of a listed parameter is intended to be open ended such that the variable may be a function of a single parameter or a plurality of parameters.

It is also noted that recitations herein of “at least one” component, element, etc., should not be used to create an inference that the alternative use of the articles “a” or “an” should be limited to a single component, element, etc.

For the purposes of describing and defining the present inventive technology it is noted that the terms “substantially” and “about” are utilized herein to represent the inherent degree of uncertainty that may be attributed to any quantitative comparison, value, measurement, or other representation. The terms “substantially” and “about” are also utilized herein to represent the degree by which a quantitative representation may vary from a stated reference without resulting in a change in the basic function of the subject matter at issue.

1. A method for preparing a self-bound hybrid catalyst, the method comprising:
mixing a powder mixture and a binder component to form a hybrid base catalyst; and
adding an impregnation solution comprising gallium to the hybrid base catalyst to form the self-bound hybrid catalyst after drying and calcination, wherein
the powder mixture comprises a metal oxide catalyst support and an 8-membered ring (8-MR) microporous catalyst component, the metal oxide catalyst support comprises zirconia, and
the binder component comprises a zirconium salt solution, a zirconium salt gel, a zirconium salt slurry, a slurry of carbonates or oxides or hydroxides of zirconium or a colloidal solution of carbonates or oxides or hydroxides of zirconium, where the binder component has a pH of from 2-8.

2. The method of claim 1, wherein the gallium is introduced into the impregnation solution via a gallium-containing precursor.

3. The method of claim 1, wherein the impregnation solution comprises at least one rare earth element.

4. The method of claim 1, wherein the impregnation solution comprises one or more of nickel, palladium, or platinum.

5. The method of claim 1, wherein the impregnation solution has a pH that is from 2 to 10.

6. The method of claim 1, wherein the impregnation solution has a pH that is from 4 to 7.

7. The method of claim 1, wherein the impregnation solution comprises a chelating or a complexing agent.

8. The method of claim 7, wherein the chelating or a complexing agent is selected from the group consisting of carboxylic acids, iminoacids, amines, glycols, and mixtures thereof.

9. The method of claim 1, wherein the impregnation solution comprises citric acid.

10. The method of claim 1, wherein binder component comprises at least one of ammonium zirconium carbonate, zirconyl acetate, zirconium oxide and zirconium hydroxide.

11. The method of claim 1, wherein the self-bound hybrid catalyst has a particle size that is from 0.5 mm to 6.0 mm.

12. The method of claim 1, wherein the self-bound hybrid catalyst, has a particle size of less than 3.0 mm.

13. The method of to claim 1, wherein the microporous catalyst component comprises SAPO-34.

14. The method of claim 1, wherein the microporous catalyst component comprises uncalcined SAPO-34.

15. A process for preparing C₂ to C₄ hydrocarbons comprising:
introducing a feed stream comprising hydrogen gas and a carbon-containing gas selected from the group consisting of carbon monoxide, carbon dioxide, and mixtures thereof into a reaction zone of a reactor; and
converting the feed stream into a product stream comprising C₂ to C₄ hydrocarbons in the reaction zone in the presence of a self-bound hybrid catalyst formed according to the method of claim 1.
